(19) United States
(12) Patent Application Publication
(10) Pub. No.: US 2006/0051302 A1
(43) Pub. Date: Mar. 9, 2006

(54) METHODS OF TREATING DERMAL/EPIDERMAL JUNCTIONS AND COSMETIC PREPARATIONS FOR USE THEREIN

(76) Inventors: Christine Jeanmaire, Nancy (FR); Gilles Pauly, Nancy (FR)

Correspondence Address: COGNIS CORPORATION;PATENT DEPARTMENT, 300 BROOKSIDE AVENUE, AMBLER, PA, 19002, US

(21) Appl. No.: 10/527,737

(22) PCT Filed: Sep. 4, 2003
(86) PCT No.: PCT/EP03/09809

§ 371 Date: Mar. 14, 2005

(30) Foreign Application Priority Data
Sep. 13, 2002 (EP) 02292247.0

(51) Int. Cl. A61K 8/97; A61K 8/73; A61K 36/064; A61K 36/26; A61K 36/48

(52) U.S. Cl. 424/59; 424/195.16; 424/74; 424/757; 424/764; 424/70.13

(57) ABSTRACT

Methods for protecting, modulating and/or improving the dermal/epidermal junctions of the skin, scalp and/or mucous membranes are described wherein a preparation comprising at least one substance which effects a modulation of one or more dermal/epidermal junction components selected from the group consisting of plectin/HD1, entactin/nidogen, percalan and combinations thereof, is applied to the skin, scalp, and/or mucous membrane. Plant extracts and other compounds useful in such methods and cosmetic preparations containing such substances are also described.

### FIELD OF THE INVENTION

This invention relates generally to cosmetic preparations and, more particularly, to a process for improving and/or for protecting the dermal/epidermal junctions of the skin, scalp and mucous membrane and for protecting the human skin against ageing, oxidative stress and against the harmful effects of environmental toxins and UV radiation. The invention also relates to the use of a substance which effects a modulation of plectin/HD1 and/or entacin/nidogen and/or perlecan for the production of cosmetic preparations for improving and/or for protecting the dermal/epidermal junctions.

### PRIOR ART

The basement membrane is a connecting cell structure between morpholigically different tissues. In the skin, it is largely found as tissue surrounding blood vessels and between the dermis and epidermis. This last region separates the epidermis and its appendages from the dermis and, accordingly, is known as a dermal/epidermal junction (DEJ). The DEJ has a complex structure consisting of hemidesmosomes, intermediate filaments, anchoring filaments, lamina densa and anchoring fibrils. The biochemical components mainly occurring therein include laminin-5 in the lamina lucida; the antigens AgBP 230 and AgPB 180 and plectin/HD1 in hemidesmosomes; entactin/nidogen and the proteoglycan perlecan in the lamina lucida and lamina densa; type IV collagen in the lamina densa and the proteoglycan type VII collagen as a constituent of the anchoring fibrils in the sub-lamina densa. These components form an interacting network.

The DEJ is the most important structure of the skin. It establishes the connection between the epidermis and the underlying dermis and maintains the integrity of the epithelial tissue by anchoring cells with the extracellular matrix via the special connecting cell complexes of the hemidesmosomes, filaments and fibrils.

On the one hand, it represents a filter for the flow of special molecules, on the other hand it enables information, for example on growth factors, to be exchanged between keratinocytes and the dermis.

The ageing-induced changes to the DEJ include the decreasing thickness of the junctions and the reduction of the cytoplasmatic cell strands consisting of basement keratinocytes in the dermis. The resulting reduction in the surface of the DEJ leads to a reduced resistance of the tissue, to a reduction in skin firmness and to an increase in the formation of wrinkles. Other signs of ageing, such as doubling of the lamina densa, ageing of the anchoring fibrils or modifications of cell components of the DEJ, also lead to a loosening of the anchorage through the epidermal system with increasing age. The ageing-induced reduction of type VII collagen is also explained by hydrolysis of the collagen through metalloproteinases. Type VII collagen appears to be less resistant to proteases with increasing age.

UV-induced damage, which leads to a reduction in the content of type VII collagen in the anchoring fibrils, also results in loosening of the bond between dermis and epidermis and hence in a reduction in elasticity of the skin and in an increase in wrinkles.

Many diseases which impair the DEJ lead to the development of sub-epidermal increases in extent. They show as a common feature a reduction in cohesion between dermis and epidermis which is manifested at the DEJ level in the formation of indentations.

In order to improve the function of the dermal/epidermal junctions, constituents of the DEJ in particular, such as lamin (FR 2813018, WO 97/48415) or kalinin (WO 92/17498), have been used in cosmetic and dermatological formulations. The stimulation of type IV collagen by magnesium aspartate (WO 99/62481), saponins (FR 2779058) or plant extracts (EP 668072) and the simulation of type VII collagen by elagic acid (WO 99/16415), plant extract of Potentilla erecta (WO 98/19664) or Bertholletia extract (U.S. Pat. No. 6,004,568) have also been disclosed for the production of cosmetic anti-ageing, anti-wrinkle and skin-firming preparations.

Nevertheless, there is a still a need for effective protection of the skin against environmentally induced ageing. Accordingly, the problem addressed by the present invention was to provide new mechanisms for improving the dermal/epidermal junctions of the skin, scalp and mucous membrane which would contribute towards delayed ageing of the skin and to protection of the skin, scalp and mucous membrane against environmental influences, oxidative stress, toxic substances or UV radiation and which could therefore be effectively used in cosmetic preparations for topical application.

### DESCRIPTION OF THE INVENTION

The present invention relates to a cosmetic treatment process for improving and/or for protecting the dermal/epidermal junctions of the skin, scalp and mucosa, characterized in that a preparation containing at least one substance which effects a modulation of plectin/HD1 and/or entactin/nidogen and/or perlecan is topically applied.

The present invention also relates to the use of a substance which effects a modulation of plectin/HD1 and/or entactin/nidogen and/or perlecan for the production of cosmetic preparations for improving and/or for protecting the dermal/epidermal junctions of the skin, scalp and mucous membrane, to the use of this substance for the production of cosmetic preparations for protection against ageing of the skin and to the use of the substance for the production of cosmetic preparations for protection against oxidative stress, the harmful effects of environmental toxins and UV radiation.

It has surprisingly been found that the modulation of molecules, such as plectin/HD1, entactin/nidogen and/or perlecan leads to the maintenance and improvement of the dermal/epidermal junctions of the skin, scalp and mucous membrane. The function of the DEJ is an essential requirement not only for health, but also for beauty care. It provides for good cohesion between the epidermis and the underlying tissues of the dermis and thus maintains the elasticity and firmness of the skin and enables wrinkling to be prevented. In addition, the DEJ on the one hand guarantees the effective nourishing of the skin through the passage of vital molecules between the epidermis and dermis and, on the other hand, affords protection against the penetration of harmful molecules into relatively deep layers of skin.

Now, the modulation of plectin/HD1, entactin/nidogen and/or perlecan by the topical application of a preparation which stimulates those substances has shown that the dermal/epidermal junction or the entire complex network of the DEJ can be strengthened in this way. This results in firming of the skin and in a reduction in wrinkle formation. Signs of ageing, including those caused by UV radiation, can be effectively prevented by the improved anchorage between the components of the DEJ and the associated increased stability and increasing elasticity of the tissue.

By virtue of the improvement in the molecule passage function, the exchange between keratinocytes and the dermis and the nourishing of the skin are optimized, so that the skin is protected not only against ageing, but also against the harmful effects of UV radiation and toxic environmental influences because the defense against harmful molecules is also strengthened by the improved nourishment.

The DEJ surrounds the hair follicles on the scalp and here, too, affords the follicles protection, so that strengthening of the DEJ in this region leads to an improvement in the properties of the hair and, in particular, is effective against hair loss or hair damage.

Accordingly, the topical cosmetic preparations which contain at least one substance that effects a modulation of plectin/HD1 and/or entactin/nidogen and/or perlecan have a preventative effect against ageing of the skin and the harmful effects of oxidative stress, environmental toxins and UV radiation on the skin, scalp, hair and mucous membrane. Besides the preventative effect, however, the modulation of the special molecules also leads to accelerated regeneration of the skin, scalp and mucous membrane after damage.

Plant extracts, more particularly the extract of Pisum sativum, Ruscus aculeatus, Centella asiatica, Calendula officinalis, Aesculus hippocastanum and/or Hibiscus esculentus, have proved to be particularly suitable modulators. However, plectin/HD1 and/or entactin/nidogen and/or perlecan can also be modulated by the administration of low molecular weight peptides which are required for the formation of the DEJ components and which have a similar sequence to constituents of plectin/HD1 and/or entactin/nidogen and/or perlecan.

Modulation of the molecules through at least one substance selected from the group consisting of mannitol, cyclodextrin, yeast extract, panthenol, propylene glycol, ammonium, glycyrrhizate and disodium succinate has also been observed. The combination of these constituents in particular contributes to an advantageous effect on the dermal/epidermal junction.

Since the DEJ is highly susceptible to degradation by a number of different proteases, the use of plant extracts, peptides and other active substances with anti-protease activity contributes significantly towards maintaining the structure. In combination with substances that effect the modulation of plectin/HD1 and/or entactin/nidogen and/or perlecan, active substances with anti-protease activity can contribute to an above-average increase in effect.

Besides these substances or plant extracts, the cosmetic preparations may additionally contain UV protection factors and/or antioxidants. The combination of substances which effect a modulation of plectin/HD1 and/or entactin/nidogen and/or perlecan with UV protection factors and/or antioxidants leads to a synergistic mode of action through the various mechanisms involved and affords excellent protection against harmful effects and ageing of the skin by UV light.

Plectin/HD1

Plectin and HD1 are synonyms for the same molecule. It is localized in the hemidesmosomes, has a molecular weight of ca. 500 dalton and serves to anchor proteins of the cytoskeleton, such as keratin, vimentin or proteins of the microtubuli.

Entactin/Nidogen

Entactin and nidogen are also different names for the same molecule. It is a glycoprotein with a molecular weight of ca. 150 dalton which consists of two terminal globular regions interconnected by a long-chain structure. This glycoprotein is critically responsible for the structural stability of the DEJ by establishing a connection between laminin and type IV collagen and contributing towards anchoring a number of other components, such as fibulin or perlecan.

Perlecan

All basement membranes contain proteoglycans. The proteoglycan mainly occurring in the DEJ is perlecan, a heparan sulfate, which is synthesized by dermal fibroblasts. Perlecan consists of a large core protein and three heparan sulfate chains. Its function is inter alia to stabilize a bond between laminin-6 and nidogen. Besides their anchoring functions, heparan sulfate proteoglycans also bind diffusing molecules, such as enzymes and growth factors, and thus may also have an influence on the behavior and properties of cells.

UV Protection Factors and Antioxidants

UV protection factors in the context of the invention are, for example, organic substances (light filters) which are liquid or crystalline at room temperature and which are capable of absorbing ultraviolet or infrared radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor;
4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzalmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.0)decane derivatives.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and enamine compounds. The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used.

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, y-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example y-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

### EXAMPLES

### Example 1

### Plectin Expression

Reagents

The monoclonal antibody, anti-plectin, and secondary antibody, fluorescein isothiocyanate (FITC)-conjugated anti-mouse antibody IgG, were obtained from Tebu and Clinisciences. PBS (phosphate buffered saline pH 7.2) and Evans blue were obtained from BioMerieux and TGF comes from Sigma.

EKIN Reconstructed Human Skin:

Reconstructed human skin from Episkin (Ekin kit) consists of a dermal support made from collagen. The keratinocytes were removed from this support. After removal of the keratinocytes and differentiation in a culture exposed to air, a human skin equivalent was obtained.

Test Substances

TGF beta was used as a positive control. The test substance used was a preparation consisting of propylene glycol, Ruscus aculeatus root extract, Centella asiatica extract, panthenol, water, Calendula officinalis flower extract, hydrolyzed yeast proteins, Aesculus hippocastanum extract and ammonium glycyrrhizate in variable percentages by weight. The reconstructed human skin was topically treated daily for three days with 0.01% of this preparation or with 10 ng/ml of TGF beta. This corresponds to a total quantity of preparation of 100 microliters per reconstructed skin. Immediately after this treatment, biopsies were taken and stored in liquid nitrogen pending evaluation.

Immunohistochemistry

Ten micrometers of the cryostat section (biopsy stored in liquid nitrogen) were fixed to glass microscope slides and stored in cold acetone for ten minutes. The section was then washed in PBS and dried in air.

The section was incubated with the monoclonal antibody, anti-plectin, for one hour at room temperature in a 1/150 solution. After washing with PBS, the section was incubated with fluorescein isothiocyanate (FITC)-conjugated antimouse antibody for 45 minutes in a 1/40 solution. Negative controls were obtained by leaving out the first antibody. After extended washing with PBS, the immuno-treated sections were treated for 10 mins. with Evans blue. The sections were examined with a Zeiss confocal laser microscope.

Quantification

The images obtained by the confocal laser microscope were converted and analyzed by mathematically morphological software (Quantimet Q500, Leica). The results were expressed as percent of the reconstructed skin surface covered with plectin (FITC).

Results

| | Control without | TGF | Preparation of the |
|---|---|---|---|
| | treatment | beta | test substance |
| % of the plectin content in | 4.16 | 21.43 | 37.9 |
| the skin section | | | |

Without the treatment, only a slight expression of the DEJ component plectin was observed in the reconstructed skin.

The topical treatment with the preparation of the test substances shows a marked increase in the expression of plectin in the reconstructed skin. The positive control TGF also shows an increase in the expression of plectin in the reconstructed skin.

### Example 2

### Perlecan Expression

Reagents

The monoclonal antibody, anti-plectin, and secondary antibody, fluorescein isothiocyanate (FITC)-conjugated anti-mouse antibody IgG, were obtained from Tebu and Clinisciences. PBS (phosphate buffered saline pH 7.2) and Evans blue were obtained from BioMerieux and TGF comes from Sigma.

Human Primary Fibroblast Culture

A cell suspension of human fibroblasts was prepared by standard digestion of collagenase from human dermis of adults obtained by plastic surgery. The fibroblasts were drawn onto glass dishes with incubation chambers and grew to coalescence in the culture medium.

Test Substances

TGF beta was used as a positive control. A hydrolyzed Hibiscus esculentus extract was used as the test substance.

The human fibroblasts were cultivated for 6 days in the presence of 0.1% of the test substance or in the presence of 10 ng/ml TGF beta in the culture medium. The perlecan expression was then evaluated by immunocytochemistry.

Immunohistochemistry

The fibroblast culture in the glass dishes was fixed for 10 minutes in cold methanol and washed with PBS. The fibroblast cultures were then incubated with the monoclonal antibody, anti-plectin, for one hour at 37° C. in a 1/150 solution. After washing with PBS, the section was incubated with fluorescein isothiocyanate (FITC)-conjugated antimouse antibody for 45 minutes in a 1/40 solution. Negative controls were obtained by leaving out the first antibody. After extended washing with PBS, the immuno-treated sections were treated for 10 mins. with Evans blue. The sections were examined with a Zeiss confocal laser microscope.

Quantification

The images obtained by the confocal laser microscope were converted and analyzed by mathematically morphological software (Quantimet Q500, Leica). The results were expressed as percent of the surface area of the fibroblast culture covered with perlecan (FITC).

Results

| | Control without | | |
|---|---|---|---|
| | treatment | TGF beta | Test substance |
| % of the perlecan content in | 1.28 | 25.07 | 8.0 |
| the fibroblast culture | | | |

Without the treatment, only a slight expression of the DEJ component perlecan was observed in the fibroblast culture.

The treatment with the test substance shows an increase in the expression of perlecanin in the fibroblast culture. The positive control TGF also shows an increase in the expression of perlecan in the fibroblast culture.

The results of Examples 1 and 2 show that the test substances (products of Laboratories Serobiologiques) can increase the expression of plectin and perlecan.

1-11. (canceled)

12. A method comprising:
(a) providing a preparation comprising at least one substance which effects a modulation of one or more dermal/epidermal junction components selected from the group consisting of plectin/HD1, entactin/nidogen, perlecan and combinations thereof; and
(b) applying the preparation to a substrate selected from the group consisting of skin, scalp, mucous membrane and combinations thereof.

13. The method according to claim 12, wherein the at least one substance comprises a plant extract.

14. The method according to claim 13, wherein the plant extract is obtained from a plant selected from the group consisting of Pisum sativum, Ruscus aculeatus, Centella asiatica, Calendula officinalis, Aesculus hippocastanum, Hibiscus esculentus, and mixtures thereof.

15. The method according to claim 12, wherein the at least one substance comprises a component selected from the group consisting of mannitol, cyclodextrin, yeast extracts, panthenol, propylene glycol, ammonium glycyrrhizate, disodium succinate, low molecular weight polypeptides, and mixtures thereof.

16. The method according to claim 12, wherein the preparation comprises two or more substances which effect a modulation of one or more dermal/epidermal junction components selected from the group consisting of plectin/HD1, entactin/nidogen, perlecan and combinations thereof.

17. The method according to claim 12, wherein the at least one substance comprises two or more plant extracts obtained from plants selected from the group consisting of Pisum sativum, Ruscus aculeatus, Centella asiatica, Calendula officinalis, Aesculus hippocastanum, Hibiscus esculentus, and mixtures thereof.

18. The method according to claim 13, wherein the preparation further comprises a component selected from the group consisting of mannitol, cyclodextrin, yeast extracts, panthenol, propylene glycol, ammonium glycyrrhizate, disodium succinate, low molecular weight polypeptides, and mixtures thereof.

19. The method according to claim 17, wherein the preparation further comprises a component selected from the group consisting of mannitol, cyclodextrin, yeast extracts, panthenol, propylene glycol, ammonium glycyrrhizate, disodium succinate, low molecular weight polypeptides, and mixtures thereof.

20. The method according to claim 12, wherein the preparation further comprises a component having anti-protease activity.

21. The method according to claim 12, wherein the preparation further comprises a component selected from the group consisting of UV protection factors, antioxidants and mixtures thereof.

22. The method according to claim 12, wherein the preparation comprises a Ruscus aculeatus root extract, a Centella asiatica extract, a Calendula officinalis flower extract, and a Aesculus hippocastanum extract.

23. The method according to claim 22, wherein the preparation further comprises a component selected from the group consisting of propylene glycol, panthenol, a hydrolyzed yeast protein, ammonium glycyrrhizate and mixtures thereof.

24. The method according to claim 12, wherein the at least one substance comprises a hydrolyzed Hibiscus esculentus extract.

25. A cosmetic preparation comprising: (a) at least one substance which effects a modulation of one or more dermal/epidermal junction components selected from the group consisting of plectin/HD1, entactin/nidogen, perlecan and combinations thereof; and (b) a component selected from the group consisting of UV protection factors, antioxidants and mixtures thereof.

26. The cosmetic preparation according to claim 25, wherein the at least one substance comprises a plant extract.

27. The cosmetic preparation according to claim 26, wherein the plant extract is obtained from a plant selected from the group consisting of Pisum sativum, Ruscus aculeatus, Centella asiatica, Calendula officinalis, Aesculus hippocastanum, Hibiscus esculentus, and mixtures thereof.

28. The cosmetic preparation according to claim 25, wherein the at least one substance comprises a component selected from the group consisting of mannitol, cyclodextrin, yeast extracts, panthenol, propylene glycol, ammonium glycyrrhizate, disodium succinate, low molecular weight polypeptides, and mixtures thereof.

29. A cosmetic preparation comprising two or more plant extracts obtained from plants selected from the group consisting of Pisum sativum, Ruscus aculeatus, Centella asiatica, Calendula officinalis, Aesculus hippocastanum, Hibiscus esculentus, and mixtures thereof.

30. The cosmetic preparation according to claim 29, wherein the two or more plant extracts comprise a Ruscus aculeatus root extract, a Centella asiatica extract, a Calendula officinalis flower extract, and a Aesculus hippocastanum extract.
